(19) United States
(12) Patent Application Publication
(10) Pub. No.: US 2010/0036219 A1
(43) Pub. Date: Feb. 11, 2010

(54) Blood Sampler

(75) Inventors: Cho-Liang Chung, Dashu Township (TW); Shian-Ching Jang, Dashu Township (TW); Chien-Chen Chen, Danshuei Township (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: I SHOU UNIVERSITY, Dashu Township (TW)

(21) Appl. No.: 12/335,366

(22) Filed: Dec. 15, 2008

(30) Foreign Application Priority Data
Aug. 5, 2008 (TW) 097129641

(51) Int. Cl. A61B 5/1468; A61B 5/15; C12Q 1/25

(52) U.S. Cl. 600/345; 600/573; 435/4

(57) ABSTRACT

A blood sampler is adapted for penetrating epidermis of skin in order to collect blood, and includes a plate, a thin-film electrode unit, and a plurality of spaced apart penetrating members. The plate has an upper surface that is indented to form a recess adapted for containing the blood. The thin-film electrode unit is provided in the plate and has a reactive section that is exposed at a bottom of the recess. The penetrating members are provided on the reactive section of the thin-film electrode unit. Each of the penetrating members has a bottom end that is wire-bonded to the reactive section of the thin-film electrode unit, and a top end that is not higher than the upper surface of the plate.

### BACKGROUND OF THE INVENTION

1. Field of the Invention

This invention relates to a sampler, more particularly to a blood sampler.

2. Description of the Related Art

Insulin that is secreted by pancreas regulates blood glucose levels in human bodies. Abnormal blood glucose metabolism that arises from insufficient secretion of insulin or insulin resistance in the human bodies may result in diabetes mellitus that is a chronic disease. Control of the blood glucose levels is critical with regard to patients with diabetes mellitus. Consequently, the patients with diabetes mellitus are required to regularly collect individual blood samples for measurement of the blood glucose levels.

Numerous tools for blood sampling, such as those disclosed in Taiwanese Application Number 095135177, US 2005/0085839, and US 2007/0173876, are available for patients with diabetes mellitus to collect the individual blood samples. Generally, each of the above tools includes a lancing mechanism for piercing or cutting epidermis of skin in order to collect blood. The measurement of the blood glucose levels can be carried out by virtue of the patients' blood.

A large size of the lancing mechanism pertaining to each of the conventional tools for blood sampling may result in a large wound. When the lancing mechanism is operated to pierce or cut the epidermis of the skin, many pain receptors (nociceptors) are stimulated and subsequently transmit signals that lead to perception of pain in response to the large wound, thereby causing the patients to feel a lot of pain. In addition, blood coagulation of the patients with diabetes mellitus is disordered. Thus, healing of the large wound is difficult.

Each of the conventional tools for blood sampling has a complicated three-dimensional structure that gives rise to reduced portability and a higher production cost. Prices of the conventional tools are increased as well. The patients prefer to reuse the tools instead of discarding the tools immediately after use of the same. Therefore, results of the measurement of the blood glucose levels are influenced. Infection may further be induced.

### SUMMARY OF THE INVENTION

The object of the present invention is to provide a blood sampler that can overcome the aforesaid drawbacks of the prior art.

According to this invention, a blood sampler is adapted for penetrating epidermis of skin in order to collect blood, and includes a plate, a thin-film electrode unit, and a plurality of spaced apart penetrating members. The plate has an upper surface that is indented to form a recess adapted for containing the blood. The thin-film electrode unit is provided in the plate and has a reactive section that is exposed at a bottom of the recess. The penetrating members are provided on the reactive section of the thin-film electrode unit. Each of the penetrating members has a bottom end that is wire-bonded to the reactive section of the thin-film electrode unit, and a top end that is not higher than the upper surface of the plate.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a top view of the first preferred embodiment of a blood sampler according to this invention;

FIG. 2 is a sectional view of the first preferred embodiment taken along line II-II in FIG. 1;

FIG. 3 is a schematic view to illustrate a gold wire that has a tip protruding outside a tip of a wire capillary used in bonding a penetrating member according to the first preferred embodiment;

FIG. 4 is a schematic view to illustrate a gold ball that is formed by virtue of melting the tip of the gold wire during the wire bonding of the penetrating member;

FIG. 5 is a schematic view to illustrate the gold ball that is welded on a thin-film electrode unit during the wire bonding of the penetrating member;

FIG. 6 is a schematic view to illustrate the penetrating member formed by the wire bonding and a portion of the gold wire protruding outside the tip of the wire capillary for subsequent wire bonding operation; and

FIG. 7 is a sectional view of the second preferred embodiment of the blood sampler according to this invention.

### DETAILED DESCRIPTION OF THE PREFERRED EMBODIMENTS

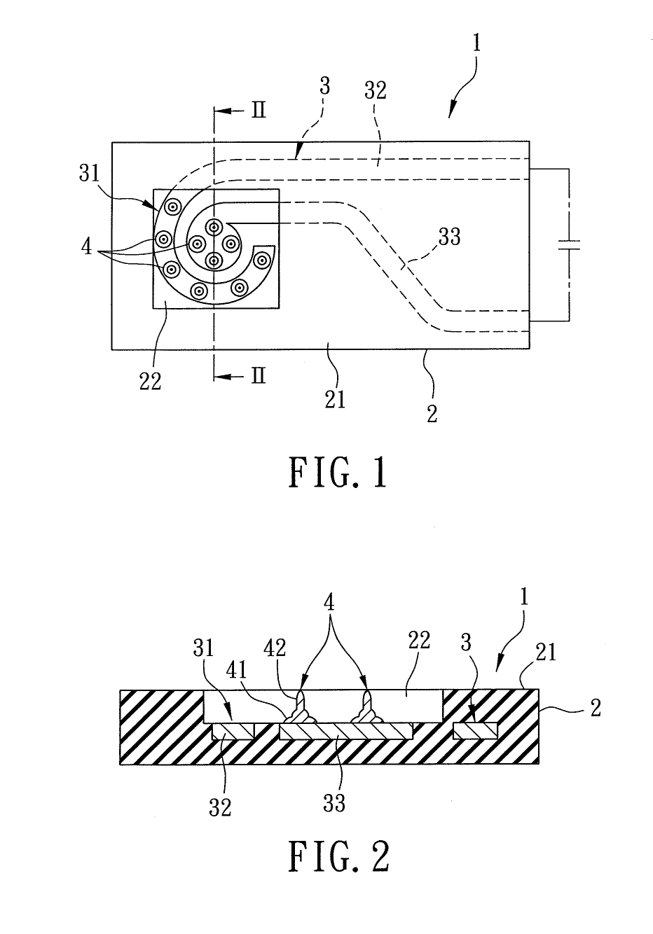

Referring to FIGS. 1 and 2, the first preferred embodiment of a blood sampler 1 according to the present invention is adapted for penetrating epidermis of skin in order to collect blood, and includes a plate 2, a thin-film electrode unit 3, and a plurality of spaced apart penetrating members 4.

The plate 2 may be made from a polymer, a piece of paper, or a metallic foil. Preferably, the plate 2 is rectangular, and has a very small thickness (about 0.5 millimeter) and an upper surface 21 that is indented to form a recess 22 adapted for containing the blood.

The thin-film electrode unit 3 is made from an electrically conductive material, and is provided in the plate 2. The thin-film electrode unit 3 has a reactive section 31 exposed at a bottom of the recess 22, a first electrode 32, and a second electrode 33 that is disposed apart from the first electrode 32. The first electrode 32 and the second electrode 33 are adapted to be electrically conducted by the blood. The thin-film electrode unit 3 can output an electrical signal at one side opposite to the reactive section 31 for measurement of blood glucose levels when the first and second electrodes 32,33 are electrically conducted.

The spaced apart penetrating members 4 are provided on the reactive section 31 of the thin-film electrode unit 3. In this embodiment, each of the penetrating members 4 is made from a gold wire, and has a bottom end that is wire-bonded to the reactive section 31 of the thin-film electrode unit 3, and a top end that is not higher than the upper surface 21. The penetrating members 4 extend from the bottom ends to the top ends thereof. Preferably, each of the penetrating members 4 includes a base 41 at the bottom end, and a needle 42 that extends from the base 41 to the top end. Instead of the gold wire, a gold-containing material may be used to make the penetrating members 4.

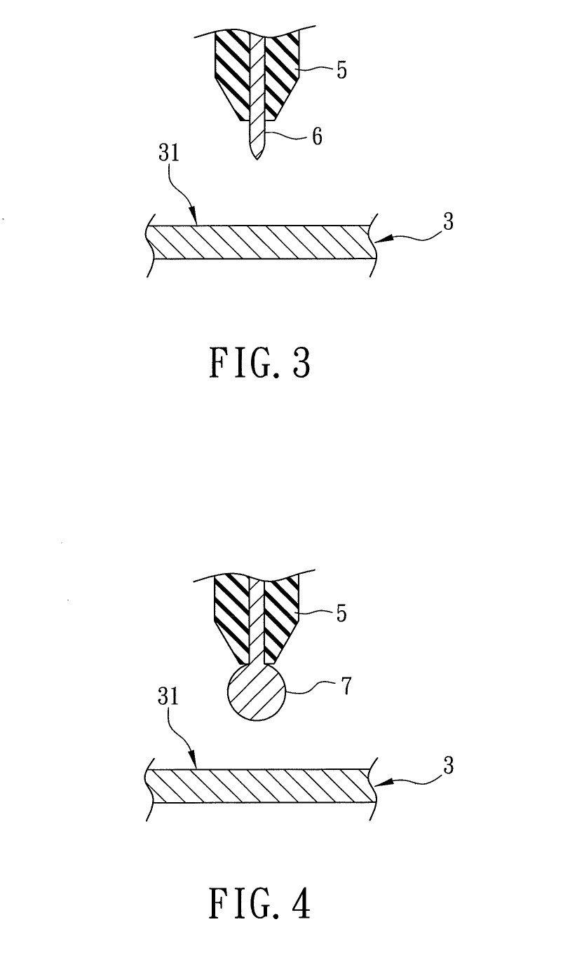

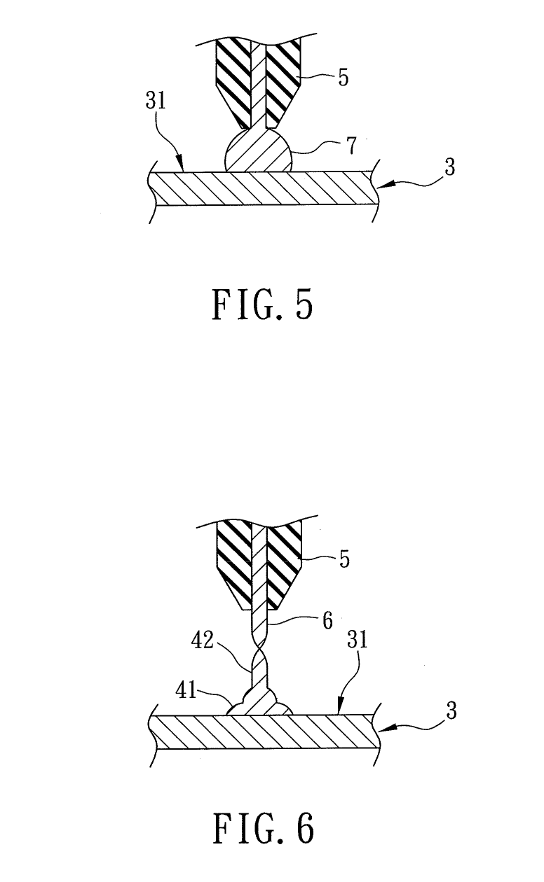

Referring to FIGS. 3, 4, 5, and 6, each of the penetrating members 4 is formed by wire bonding. A tip of a gold wire 6 protrudes outside a tip of a wire capillary 5 (shown in FIG. 3). The tip of the gold wire 6 is melted to form a gold ball 7 via ultrasonic arc welding (shown in FIG. 4). Volume of the gold ball 7 is the same as that of the tip of the gold wire 6. The wire capillary 5 is lowered so as to press the gold ball 7 against the reactive section 31 of the thin-film electrode unit 3 (shown in FIG. 5). Ultrasonic friction welding is conducted to weld the gold ball 7 on the reactive section 31 of the thin-film electrode unit 3. The base 41 of one of the penetrating members 4 is almost formed. After the gold ball 7 is welded on the reactive section 31, the wire capillary 5 is controlled to release a portion of the gold wire 6 and to move upward so that the portion of the gold wire 6 is pulled upwardly and caused to break at the interface in the heat-affected zone having thick crystal lattice. During the pulling operation, the gold wire 6 is gradually thinned due to its ductility, thereby forming the base 41 and the needle 42 and leaving a length of the gold wire 6 protruding out of the wire capillary 5 for subsequent forming of the next penetrating member 4.

From the above-described forming operation, it can be understood that the smallest distance between adjacent ones of the needles 42 is limited by the width of the base 41. In particular, the smallest distance between adjacent ones of the needles 42 of the penetrating members 4 may be substantially equal to the diameter of the base 41. Distribution of pain receptors of the skin, a maximum width of the needle 41 (i.e., a maximum width of the gold wire 6), and a production yield resulting from the wire bonding should be considered in order to decide an optimum value for the distance between adjacent ones of the penetrating members 4. The production yield resulting from the wire bonding varies with the maximum width of the gold wire 6 and the distance between adjacent ones of the penetrating members 4. Generally, the finer the gold wire 6 and the smaller the distance, the lower will be the production yield for good quality products.

Preferably, the distance between adjacent ones of the penetrating members 4 ranges from 10 to 300 micrometers. The needle 42 of each of the penetrating members 4 has the maximum width that is equal to the maximum width of the gold wire 6 and that ranges from 20 to 100 micrometers, and a length that ranges from 500 to 2000 micrometers depending on a thickness of the epidermis of the skin and the depth of the recess 22. Each of the penetrating members 4 has a length that extends from the bottom end to the top end, and that conforms to a depth of the recess 22.

When the blood sampler 1 is used to collect the blood from a finger (or a desired body portion) of a user, the finger is positioned above the recess 22 and subsequently pressed against the penetrating members 4. The needles 42 are used for penetrating the epidermis of the skin, thereby penetrating blood vessels. The blood that flows from the blood vessels is collected and contained in the recess 22. Therefore, the first and second electrodes 32,33 are electrically interconnected by virtue of the blood. The electrical signal is transmitted from the thin-film electrode unit 3 for the measurement of the blood glucose levels.

Due to the small distances between adjacent ones of the penetrating members 4, and the small width and length of the needles 42, an amount of pain receptors that are stimulated by the blood sampler 1 is very small compared to an amount of the pain receptors that are stimulated by the conventional tools for blood sampling. Consequently, very little pain is produced. Furthermore, a wound that arises from the penetrating members 4 is very small and can easily heal compared to the large wound that arises from the lancing mechanisms of the conventional tools.

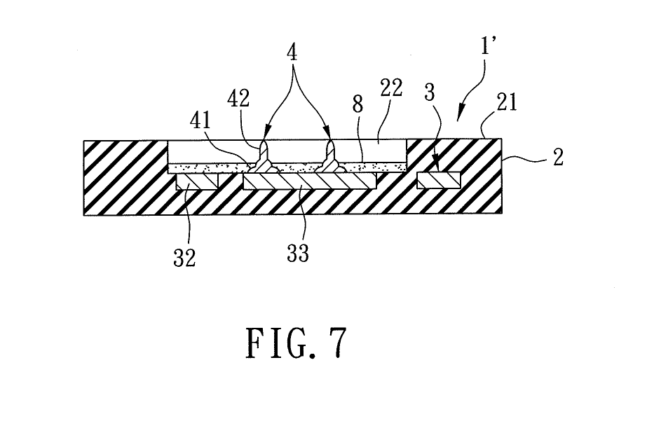

Referring to FIG. 7, the second preferred embodiment of the blood sampler 1′ according to this invention is illustrated. The structure of this preferred embodiment is similar to the structure of the first preferred embodiment. The difference between this preferred embodiment and the first preferred embodiment resides in that the blood sampler 1′ further includes an enzyme 8 provided in the recess 22 and adapted for reacting with the blood so as to change color. Change of color may indicate sufficiency of the blood collected in the recess 22, an anticipated range of the blood glucose levels, etc. Since the feature of the invention does not reside in the enzyme 8, which is known in the art, further details of the same are omitted herein for the sake of brevity.

As the wire bonding is a well-developed technique in the field of packaging technology, the fabrication of the penetrating members 4 by wire bonding can reduce production cost of the blood sampler 1,1′. A price for the blood sampler 1,1′ is further reduced such that the blood sampler 1,1′ is disposable after use of the same. Possibility of infection is decreased accordingly.

In view of a small size of each of the penetrating members 4, the thickness of the plate 2 can be reduced to a range from 0.2 to 2 millimeters when needed. The thickness of the plate 2 may be as small as that of a piece of paper having a heavier weight. As a whole, the plate 2 has a portable two-dimensional structure. Patients with diabetes mellitus can carry the blood sampler 1,1′ easily in order to collect blood in any occasion.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. A blood sampler adapted for penetrating epidermis of skin in order to collect blood, said blood sampler comprising:
a plate having an upper surface that is indented to form a recess adapted for containing the blood;
a thin-film electrode unit provided in said plate and having a reactive section that is exposed at a bottom of said recess; and
a plurality of spaced apart penetrating members provided on said reactive section of said thin-film electrode unit, each of said penetrating members having a bottom end that is wire-bonded to said reactive section of said thin-film electrode unit, and a top end that is not higher than said upper surface of said plate.

2. The blood sampler as claimed in claim 1, wherein said plate is made from a polymer, apiece of paper, or a metallic foil.

3. The blood sampler as claimed in claim 1, wherein said thin-film electrode unit includes a first electrode and a second electrode that is disposed apart from said first electrode, said first electrode and said second electrode being adapted to be electrically conducted by the blood.

4. The blood sampler as claimed in claim 1, wherein said penetrating members are made from gold or a gold-containing material.

5. The blood sampler as claimed in claim 1, wherein each of said penetrating members includes a base at said bottom end, and a needle extending from said base to said top end.

6. The blood sampler as claimed in claim 5, wherein adjacent ones of said needles have therebetween a smallest distance that is equal to a diameter of said base.

7. The blood sampler as claimed in claim 5, wherein said needle of each of said penetrating members has a width that ranges from 20 to 100 micrometers and a length that ranges from 500 to 2000 micrometers.

8. The blood sampler as claimed in claim 1, further comprising an enzyme that is provided in said recess and that is adapted for reacting with the blood so as to change color.
